(19) United States
(12) Patent Application Publication
(10) Pub. No.: US 2022/0215956 A1
(43) Pub. Date: Jul. 7, 2022

(54) SYSTEM AND METHOD FOR IMAGE ANALYSIS USING SEQUENTIAL MACHINE LEARNING MODELS WITH UNCERTAINTY ESTIMATION

(71) Applicant: SHENZHEN KEYA MEDICAL TECHNOLOGY CORPORATION, Shenzhen (CN)

(72) Inventors: Bin Kong, Charlotte, NC (US); Youbing Yin, Kenmore, WA (US); Xin Wang, Seattle, WA (US); Yi Lu, Seattle, WA (US); Qi Song, Seattle, WA (US)

(73) Assignee: SHENZHEN KEYA MEDICAL TECHNOLOGY CORPORATION, Shenzhen (CN)

(21) Appl. No.: 17/567,486

(22) Filed: Jan. 3, 2022

Related U.S. Application Data
(60) Provisional application No. 63/134,172, filed on Jan. 5, 2021.

(51) Int. Cl. G16H 50/20; G06N 5/04; G06V 10/774; G06T 7/00

(52) U.S. Cl. CPC G16H 50/20; G06N 5/045; G06T 2207/30048; G06T 7/0012; G06V 10/774

(57) ABSTRACT

The disclosure relates to a system and method for predicting physiological-related parameters based on a medical image. The method includes receiving a medical image acquired by an image acquisition device and predicting a sequence of physiological-related parameters at a sequence of positions and simultaneously estimating an uncertainty level of the predicted sequence of physiological parameters from the medical image by using a sequential learning model. The sequential learning model is trained to minimize a loss function associated with the uncertainty level. The method not only provides predictions but also the corresponding uncertainty estimations by using sequential learning model(s), thus improving the transparency and explainability of the sequential learning model.

### CROSS REFERENCE TO RELATED APPLICATION

This application is based on and claims the benefit of priority of U.S. Provisional Application No. 63/134,172, filed on Jan. 5, 2020, which is incorporated herein by reference in its entirety.

### TECHNICAL FIELD

The present disclosure relates to a system and method for medical image analysis, and especially relates to a system and method for analyzing a medical image using a sequential learning model with uncertainty estimation.

### BACKGROUND

Sequential machine learning models have been used as essential tools to model complex sequential correlations across different domains, such as medical image analysis. Although these sequential learning models, especially state-of-the-art deep learning models such as recurrent neural networks (RNNs), can accurately convert one natural language into another or predict a sequence of physiological parameters at a plurality of anatomic structural positions, one of the challenges with such sequential learning models is the explainability. These models are often treated as black boxes and hard to decipher.

With the increasing complexity of learning models, it may improve the prediction ability for various complex problems in real practice. Nevertheless, more complex models often mean less transparency/explainability. This leads to great concerns in their deployment in their real-world deployment, especially those with significant implications such as healthcare.

One way to avoid this problem is to use simpler and more explainable models such as Gaussian processes and linear regression. However, it significantly limits the model performance.

Therefore, there is an unmet need to improve the sequential learning model, especially for those intend to model complex functions in the technical field of healthcare.

### SUMMARY

The present disclosure is provided to solve the above-mentioned problems existing in the prior art. The disclosed system and method analyze images, such as medical images using an improved sequential learning model, which not only provides a sequence of accurate predictions but also the corresponding uncertainty estimations. With the improved transparency and explainability of the sequential learning model, the disclosed system and method can improve the accuracy of its prediction process (e.g., to predict physiological-related parameters) when analyzing a medical image.

According to a first aspect of the present disclosure, it provides a method for predicting physiological-related parameters based on a medical image. The method may include receiving a medical image acquired by an image acquisition device and predicting, by a processor, a sequence of physiological-related parameters at a sequence of positions and simultaneously estimating an uncertainty level of the predicted sequence of physiological parameters from the medical image by using a sequential learning model. The sequential learning model is trained to minimize a loss function associated with the uncertainty level.

According to a second aspect of the present disclosure, it provides a system for predicting physiological-related parameters based on a medical image. The system includes a communication interface configured to receive a medical image acquired by an image acquisition device. The system may also include a processor configured to predict a sequence of physiological-related parameters at a sequence of positions and simultaneously estimating an uncertainty level of the predicted sequence of physiological parameters from the medical image by using a sequential learning model. The sequential learning model is trained to minimize a loss function associated with the uncertainty level.

According to a third aspect of the present disclosure, it provides a non-transitory computer storage medium having computer executable instructions stored thereon, wherein the computer-executable instructions, when executed by a processor, perform a method for predicting physiological-related parameters based on a medical image. The method may include acquiring a medical image acquired by an image acquisition device and predicting a sequence of physiological-related parameters at a sequence of positions and simultaneously estimating an uncertainty level of the predicted sequence of physiological parameters from the medical image by using a sequential learning model. The sequential learning model is trained to minimize a loss function associated with the uncertainty level.

The disclosed systems and methods not only provide a sequence of accurate predictions but also the corresponding uncertainty estimations simultaneously by using sequential learning model(s), so as to improve the transparency and explainability of the sequential learning model and the process of predicting physiological-related parameters from a medical image using such a model.

It should be understood that the foregoing general description and the following detailed description are only exemplary and illustrative, and do not intend to limit the claimed invention.

### BRIEF DESCRIPTION OF THE DRAWINGS

In the drawings that are not necessarily drawn to scale, similar reference numerals may describe similar components in different views. Similar reference numerals with letter suffixes or different letter suffixes may indicate different examples of similar components. The drawings generally show various embodiments by way of example and not limitation, and together with the description and claims, are used to explain the disclosed embodiments. Such embodiments are illustrative and are not intended to be exhaustive or exclusive embodiments of the method, system, or non-transitory computer-readable medium having instructions for implementing the method thereon.

FIG. 1 illustrates a flow chart of the method for predicting physiological-related parameters and the uncertainty level thereof based on a medical image, according to an embodiment of the present disclosure.

FIG. 2(a) illustrates an application example of allocation of human resources for evaluation of the prediction results from the machine learning system based on uncertainty estimation, according to an embodiment of the present disclosure.

FIG. 2(b) illustrates a comparison example of allocation of human resources for evaluations of the prediction results from the machine learning system without using the uncertainty estimation.

FIG. 3 illustrates a schematic diagram of a method for data uncertainty estimation using a sequential learning model, according to an embodiment of the present disclosure.

FIG. 4 illustrates a schematic diagram of a method for data uncertainty estimation using a sequential learning model, according to another embodiment of the present disclosure.

FIG. 5 illustrates a schematic diagram of a method for model uncertainty estimation, according to an embodiment of the present disclosure.

FIG. 6 illustrates a schematic diagram of the method for model uncertainty estimation as illustrated by FIG. 5 using latent variables, according to an embodiment of the present disclosure.

FIG. 7 illustrates a training process of a sequential learning model for both prediction of physiological-related parameters and estimation of the prediction uncertainty, according to an embodiment of the present disclosure.

FIG. 8 illustrates a testing process of a trained sequential learning model for both prediction of physiological-related parameters and estimation of the prediction uncertainty, according to an embodiment of present disclosure.

FIG. 9 illustrates a schematic block diagram of a system for predicting physiological-related parameters and the uncertainty level thereof based on a medical image, according to an embodiment of the present disclosure.

### DETAILED DESCRIPTION

Reference will now be made in detail to exemplary embodiments, examples of which are illustrated in the drawings.

Consistent throughout this disclosure, a physiological-related parameter may be any form of parameter associated with a physiological status of a subject (e.g., a human or an animal). As an example, the physiological-related parameter may take the form of class labels, continuous variables (parameter measurements), segmentation mask, etc. Besides, the physiological status may be any one of physiological functional status, physiological anatomical status (e.g., belonging to which body site, which organ, which tissue), lesion (e.g., vessel plaque, myocardial bridge, hemangioma, etc.) or non-lesion, with or without foreign matters (such as implant, stent, cannula, catheter, guide wire), etc. or its combination. As another example, the physiological-related parameter may be at least one vessel functional parameter out of blood pressure, blood velocity, blood flow-rate, wall-surface shear stress, fractional flow reserve (FFR), microcirculation resistance index (IMR), and instantaneous wave-free ratio (iFR) and/or its change parameter compared with adjacent position at one or more positions along the centerline of the vessel. Consistent throughout the disclosure, a sequence may include one or more elements, and may be distributed in temporal or spatial domain. As an example, a sequence of positions may include a single position, multiple positions distributed in a tree structure, or multiple positions at a sequence of timing points, etc.

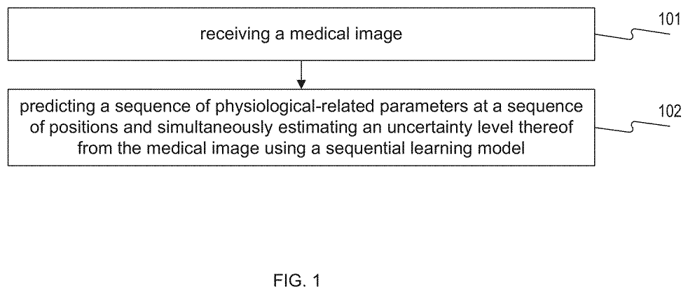

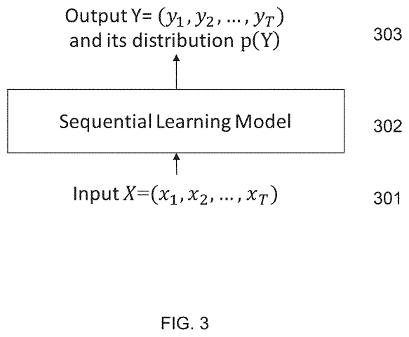

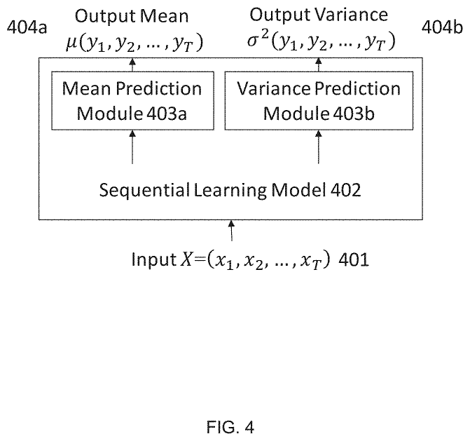

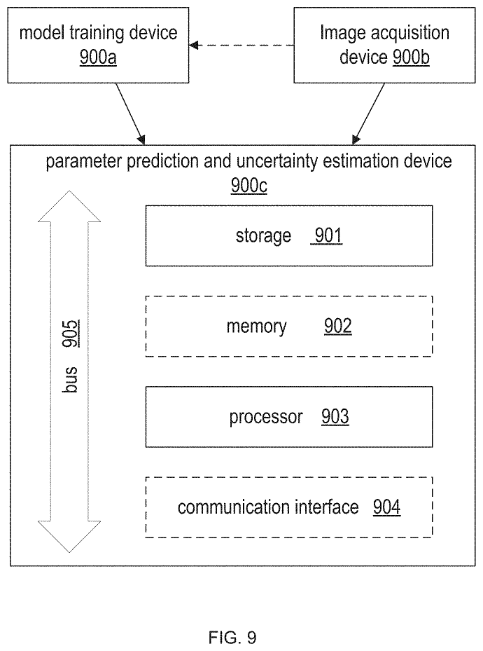

FIG. 1 illustrates a flow chart of the method for predicting physiological-related parameters and the uncertainty level thereof based on a medical image, according to an embodiment of the present disclosure. As shown in FIG. 1, the method begins at step 101: receiving a medical image. The medical image may be acquired by an image acquisition device, as will be described later (see FIG. 9). The medical image may contain the site/organ/tissue of interest, such as but not limited to brain image, cardiovascular image, etc. The cardiovascular image may be adopted as an example hereinafter. However, the embodiments disclosed in the context of the cardiovascular image may also be adapted and utilized for other medical images. In some embodiments, the cardiovascular image may be coronary CT, MRI, ultrasound, etc. Based on the acquired cardiovascular image, centerline points may be extracted and thus the image patches or feature vectors may be extracted from the centerline points. A sequence of image patches or feature vectors at a sequence of centerline points may be fed into a sequential learning model as input X (see FIG. 3 and FIG. 4). For different problems, inputs X may be image patches or features vectors extracted from the medical image, especially from a sequence of positions therein. For example, in the task of predicting physiological parameters for the vessel centerline points from cardiovascular images, the input sequence X may be image/mask patches, hand-crafted features, or other features (e.g., automatically extracted features by neural networks) extracted for the centerline points from the cardiovascular images or vessel masks.

At step 102, a sequential learning model may be applied to the medical image, e.g., the extracted sequence of image patches or feature vectors at a sequence of centerline points, to predict a sequence of physiological-related parameters Y at a sequence of positions meanwhile estimate an uncertainty level thereof. Apart from predicting the output sequence Y=(y₁, y₂, . . . , y_{T}), its uncertainty level is also estimated. Furthermore, the sequential learning model may be a single sequential learning model that performs the dual functions: predicting Y and estimating its uncertainty level. For example, the single sequential learning model may have two branches of outputs, where one outputs the sequence of physiological-related parameters while the other outputs the uncertainty level thereof. The output sequence of physiological-related parameters may be a single prediction value, or multiple values in spatial/temporal sequences, tree structures, or other data structure. In some embodiments, the output sequence Y may include any one of class labels, continuous physiological parameters, or segmentation masks at the sequence of positions or a combination thereof. Still in the task of predicting physiological parameters for the vessel centerline points from cardiovascular images, the output sequence Y may include any one of vessel physiological-functional status, blood pressure, pressure drop, blood velocity, blood flow-rate, wall shear stress, fractional flow reserve (FFR), FFR change between adjacent vessel centerline points, instantaneous wave-free ratio (iFR), and iFR change between adjacent vessel centerline points, or a combination thereof. In some embodiments, the uncertainty level may measure the uncertainty of the whole sequence, or partial segments, or several locations/points in sequences. In some embodiments, the uncertainty level may measure the uncertainty of just a single prediction. In some embodiments, the uncertainty level may take various forms, such as but not limited to variance and quartile of the distribution, conditional probability, etc. In some embodiments, the sequential learning model may include RNN models such as long short-term memory (LSTM), gated recurrent unit (GRU), and transformers.

With the automatic analysis method as shown in FIG. 1, not only the prediction result of the sequence of physiological-related parameters at the sequence of positions but also the uncertainty level thereof, are obtained simultaneously by a single sequential learning model. In some embodiments, the estimation of the uncertainty level may cover two major sources of uncertainty, data uncertainty and model uncertainty. Thus, it may improve the transparency and explainability of the sequential learning model together with its prediction process (from the input data to the output data as prediction result of the sequence of physiological-related parameters at a sequence of positions).

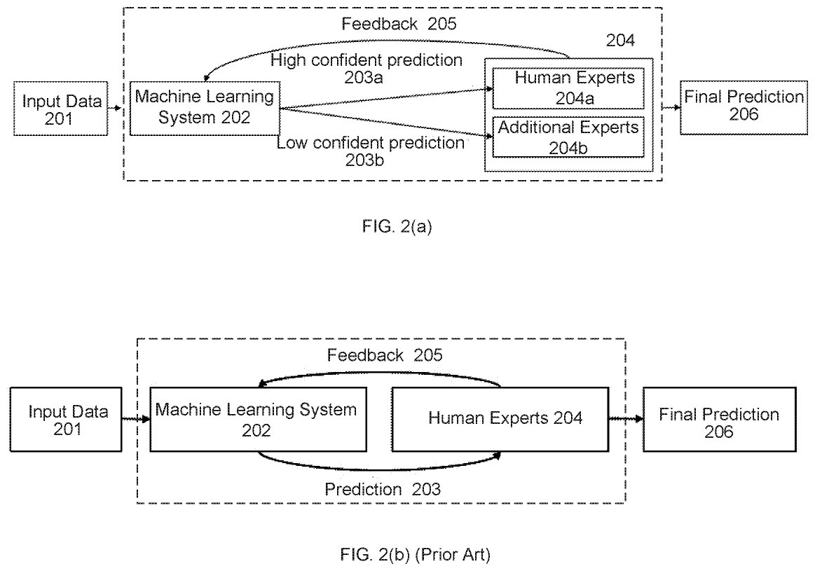

The uncertainty level may be extremely valuable for making reliable decision and improving model performance. For example, FIG. 2(a) illustrates an application example of allocation of human resources for evaluation of the prediction results from the machine learning system using uncertainty estimation, according to an embodiment of the present disclosure. As shown in FIG. 2(a), the input data 201 is firstly fed into a trained machine learning system 202 (e.g., the sequential learning model mentioned above). Simultaneously with the prediction, the machine learning system 202 also estimates the uncertainty level of the prediction, such as being high confident or low confident. The prediction may then be directed to the human experts 204 for evaluation. The human resources (such as the human experts 204) may be efficiently allocated for evaluation of the predicted sequence of physiological-related parameters based on the estimated uncertainty level thereof. Specifically, the lower the uncertainty level is, the more human resources may be allocated for evaluation. As an example, the uncertainty level of the prediction may be categorized as high confidence (see high confident prediction 203a) and low confidence (see low confident prediction 203b).

Specifically, the human resources can be divided into a smaller group of human experts 204a and a larger group of additional human experts 204b (which contains more experts than the human experts 204a). Human experts 204a are allocated to evaluate easier cases that have high confident predictions while additional experts 204b are allocated to hard cases that have low confident predictions. If the human experts, e.g., the human experts 204a or the additional experts 204b confirm the prediction result generated by machine learning system 202, the final prediction 206 is generated. Note that the feedback 205 regarding the evaluation from the human experts 204 may be provided back to the machine learning system 202 to refine the predictions. This machine-human interaction may be repeated for several iterations to maximally leverage the power of machine learning system 202. The limited human resources for evaluation of predictions are one of the bottlenecks for utilization of the machine learning system 202 in medical image analysis and computer-assisted diagnosis. By means of efficient allocation of human resources to evaluation based on the certainty level of the model-generated prediction result, the limited human resource may focus on the low confident predictions, so as to improve the accuracy of the final prediction and improve the efficiency of machine learning system 202. As a comparison, in the prior art example of FIG. 2(b), the machine learning system 202 only outputs prediction 203 without estimating the uncertainty level. Therefore, the workflow just directs the prediction 203 to the entire group of human experts 204, regardless of its confidence level. In this manner, the low confident prediction may occupy substantial bandwidth of the human resources for evaluation and therefore inefficient.

The sequential learning model may be trained to estimate the uncertainty level simultaneously with the prediction. In some embodiments, the estimation of the uncertainty level adds a Bayesian aspect to the sequential prediction problem, which may be used to integrate prior knowledge in the inference stage for prediction the sequence of physiological-related parameters at a sequence of positions. The integration of uncertainty estimation in the single sequential learning model during the training stage improves the accuracy of the prediction and the transparency and explainability during the inference stage.

In some embodiments, the predicted sequence of physiological-related parameters and the estimated uncertainty level thereof may be displayed associated with each other (i.e., in an associated manner) on the display, so as to enable a user to make a further decision. Particularly, in view of the prediction result and its uncertainty level, the user may decide to discard the prediction result (e.g., if the uncertainty level is lower than a first threshold level), add evaluation comments to the prediction result (e.g., if the uncertainty level is higher than the first threshold level but lower than a second threshold level) and transfer the same to his/her superior with higher professional level for double check, or directly approve the prediction result (with uncertainty level higher than the second threshold level).

There are various methods to model uncertainty estimation in the sequential learning model. In some embodiments, the uncertainty may be modeled by explicitly modeling the conditional probability. The uncertainty estimation takes two major sources of uncertainty into consideration. One major source originates from the input data X. This type of uncertainty may be due to the noisy measurement or limited training data. As a result, the trained learning model may not be confident for an unseen test data out of the scope of the distribution of the training data. Another major source originates from model specification. For instance, a complex sequential learning model such as RNN may easily overfit the noise in the training data. As a result, sequential learning models trained with different initializations may yield totally different prediction results. In some embodiments, sequential learning models may be provided separately for the prediction of physiological-related parameters and the estimation of the uncertainty level thereof based on the common input. In some embodiments, a single sequential learning model may include two functional branches for (1) the prediction of physiological-related parameters and (2) the estimation of the uncertainty level thereof. Accordingly, two outputs are provided by the single sequential learning model: one for the prediction of physiological-related parameters while the other for the estimation of the uncertainty level thereof.

As an example, in case that the physiological-related parameters are class labels, the method further includes predicting a sequence of class labels together with a sequence of the conditional probabilities at the sequence of positions by using the single sequential learning model. Then the uncertainty level of the predicted sequence of class labels may be estimated based on the sequence of conditional probabilities at the sequence of positions. Besides, as shown in FIG. 3, the output 303 of the sequential learning model 302 is not limited to class labels, it may include the predicted sequence of physiological parameters Y=(y₁, y₂, . . . , y_{T}), which may be the physiological parameters from the vessel centerline points, where T represents the number of the centerline points. Correspondingly, the input X 301 of the sequential learning model 302 may be a sequence X=(x₁, x₂, . . . , x_{T}) as shown in FIG. 3, which may be the patches/feature maps along the centerline points which are extracted from the cardiovascular image. As shown in FIG. 3, the output 303 of the sequential learning model 302 may also include a distribution p(Y) of Y given the input sequence X, which may be the conditional probability p(Y|X)=p(y₁, y₂, . . . , y_{T}|x₁, x₂, . . . , x_{T}). Instead of generating a single point value yₜ for each t, wherein t may be 1, 2, . . . , T, the conditional probability p(Y|X)=p(y₁, y₂, . . . , y_{T}|x₁, x₂, . . . , x_{T}) given the input sequence X=(x₁, x₂, . . . , x_{T}) may be approximated by the sequential learning model 302. For each position t, an individual uncertainty level may be estimated based on the conditional probability at position t. The higher the conditional probability at position t, the lower the corresponding uncertainty level at position t. As another example, a representative uncertainty level may be estimated for the predicted sequence of physiological parameters, Y=(y₁, y₂, . . . , y_{T}), or a part thereof, by e.g., averaging operations, minimum operations, etc. In this manner, the ground truth of the representative uncertainty level and the sequence of physiological parameters may be utilized to train the sequential learning model 302. Compared to the ground truth values of the uncertainty level at a sequence of positions, the ground truth values of the representative uncertainty level are much easier to obtain, thus facilitating the acquisition of the set of training data and thus simplifier the training of the sequential learning model 302.

In some embodiments, in case that the physiological-related parameters are continuous physiological parameters, such as FFR, as shown in FIG. 4, the method may further include predicting the sequences of mean physiological parameters μ(y₁, y₂, . . . , y_{T}) 404a together with variances σ²(y₁, y₂, . . . , y_{T}) 404b at the sequence of positions by using the single sequential learning model 402. The sequential learning model 402 may include a mean prediction module 403a for predicting the sequences of mean physiological parameters μ(y₁, y₂, . . . , y_{T}) 404a as well as a variance prediction module 403b for estimating the variances σ² (y₁, y₂, . . . , y_{T}) 404b. For instance, the mean prediction module 403a and the variance prediction module 403b may be provided as parallel output layers (such as separate branches of activation functions and fully connected layers) following a common feature extraction portion. Both modules 403a and 403b receive X=(x₁, x₂, . . . , x_{T}) as input 401.

The sequences of mean physiological parameters μ(y₁, y₂, . . . , y_{T}) 404a and the variances σ² (y₁, y₂, . . . , y_{T}) 404b may be used to approximate and determine the conditional probability assuming a Gaussian probability distribution. Under this assumption, the predicted sequences of mean physiological parameters at the sequence of positions, μ(y₁, y₂, . . . , y_{T}), may be output directly as the predicted sequence of physiological-related parameters; and the uncertainty level of the predicted sequence of physiological-related parameters may be determined based on the sequences of variances at the sequence of positions, σ² (y₁, y₂, . . . , y_{T}). During the testing stage, the sequential learning model 402 may yield larger variances for uncertain predictions, and vice versa. The uncertainties, together with the predictions, may then be used for further decision making.

In some embodiments, the sequential learning model 402 may be trained in consideration of the uncertainty level in the loss function.

For example, in case that the physiological-related parameters to be predicted are continuous physiological parameters, the cost function may include a squared L2 norm loss term L based on the sequence of variances at the sequences of positions σ²(y₁, y₂, . . . , y_{T}) and a divergence between the sequence of mean physiological parameters μ(y₁, y₂, . . . , y_{T}) and the sequences of ground truth physiological parameters Ŷ(ŷ₁, ŷ₂, . . . , ŷ_{T}). Particularly, the loss term L may be defined by equation (1):

$$L = {\sum }_{t = 1}^{T} {( \frac{μ ( {y}_{t} \vert X ) - {\hat{y}}_{t}}{{σ}^{2} ( {y}_{t} \vert X )} )}^{2} , \qquad equation ( 1 )$$

During the training stage, L may be minimized by either minimizing the difference between the prediction result Y and the ground truth Ŷ or maximizing the variance σ₂(Y|X).

In case that the physiological-related parameters are class labels, the conditional probability may be directly used in the loss function to train the sequential learning model. In some embodiments, the prediction process may be treated as a task of K-class classification, and the loss function may be defined to use the predicted sequence of probabilities at the sequence of positions therein, to minimize the divergence between the conditional probability and the corresponding label. As an example, the loss function may be defined by equation (2):

L=Σₜ₌₁^{T}Σₖ₌₁^{K}y_{t,k} log(p_{t,k|X}), equation (2)

where y_{t,k} represents the class label at the position t, and p_{t,k|X} represents the conditional probability of the class label at the position t given input as x. The loss function as defined by equation (2) is designed to minimize the divergence between the conditional probability p_{t,k|X} and the label y_{t,k}. During the testing stage, the conditional probability p_{t,k|X} output by the sequential learning model may be used directly as the uncertainty estimate for the k^{th} class.

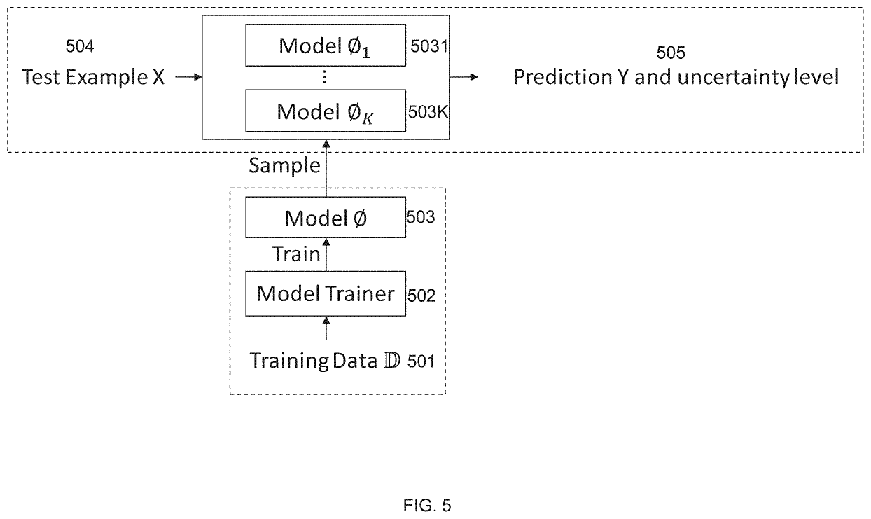

In some embodiments, the conditional probability p(Y|X) may be implicitly modeled using latent variables Z, which may capture the uncertainties in the prediction process. The uncertainty regarding the prediction Y may be approximated by sampling the latent variable Z. As shown in FIG. 5, the training data D 501 may be fed to the model trainer 502, so that the model trainer 502 may perform training on Model Ø 503 (e.g., a single sequential learning model) as well as the set of Models, i.e., Model Ø₁ 5031, . . . , Model Ø_{K}, which are obtained by randomly sampling the latent variables in Model Ø 503 for K times, where k is the number of randomly sampling. For each time of sampling, i.e., for Model Øₘ 503k, m=1, . . . ,K, based on the test example X 504, a corresponding sequence of physiological-related parameters Y^{m} may be determined. Let Y^{m}=(y₁^{m}, y₂^{m}, . . . , y_{T}^{m}) be the prediction of the m^{th} sampling procedure, the prediction variance of yₜ may be approximated by the sample variance as defined by equation (3):

$$( {y}_{t} ) = \frac{1}{K} {\sum }_{m = 1}^{K} {( {y}_{t}^{m} - ( {y}_{t} ) )}^{2} , \qquad equation ( 3 )$$

where [symbol] (yₜ) represents the prediction variance of yₜ. And the sample mean [symbol] (yₜ) may be calculated by equation (4):

$$( {y}_{t} ) = \frac{1}{K} {\sum }_{m = 1}^{K} {y}_{t}^{m} , \qquad equation ( 4 )$$

Then, the sequence of mean of the physiological-related parameters predicted for K times, i.e., [symbol] (y₁, . . . , y_{T}), may be determined as the predicted sequence of physiological-related parameters Y. Further, the sequence of variances of the physiological-related parameters predicted for K times, i.e. [symbol] (y₁, . . . , y_{T}), may be determined and the uncertainty level may be estimated based on the determined sequence of variances.

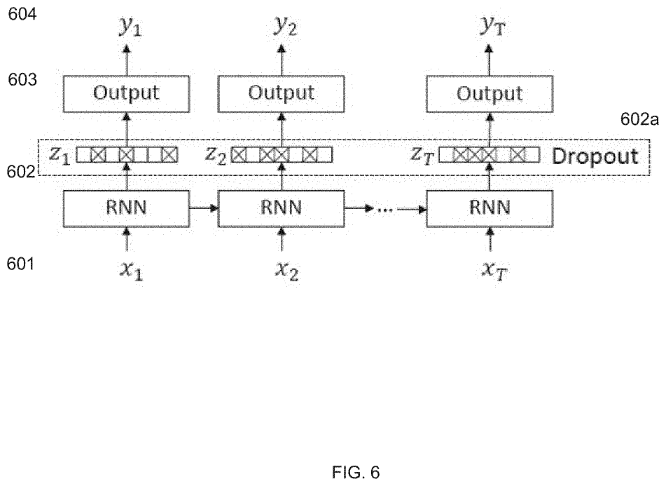

In some embodiments, the sequential learning model may be a RNN model and the above process in FIG. 5 may be adapted to RNN model to estimate the variance of the predictions therein. An RNN model includes one or more layers having multiple neurons. As an example, during the sampling procedures, one or more elements in the neurons in one or more layers of the RNN model may be dropped out, as shown in FIG. 6. The neurons in these layers may be treated as random variables Z and the network may be trained by sampling Z, such as but not limited to dropping out Z, therefore, capturing the uncertainties in the dataset. The input sequence 601 X=(x₁, x₂, . . . , x_{T}) may be fed into a sequence of RNN units 602, each of which may contain one or more layers 602a as shown by the dotted line. The neurons in these layers may be treated respectively as latent variables z₁, z₂, . . . , z_{T}, and elements (e.g., those crossed-out in RNN units 602 of FIG. 6) may be randomly sampled and dropped out. In some embodiments, during the training stage, the crossed-out elements in the latent variables z₁, z₂, . . . , z_{T} may be randomly selected and set to 0.

In the testing stage, the variance of the prediction 604 Y=(y₁, y₂, . . . , y_{T}) may be approximated by sampling Z. Specifically, for the input sequence X=(x₁, x₂, . . . , x_{T}), K predictions may be obtained by randomly dropping out Z for K times. As an example, the RNN unit 602 may be an LSTM or GRU unit. The output layers 603 may be a sequence of fully connected layers or other networks. In some embodiments, the crossed-out elements in the latent variables may be randomly selected and set to 0 during the inference stage.

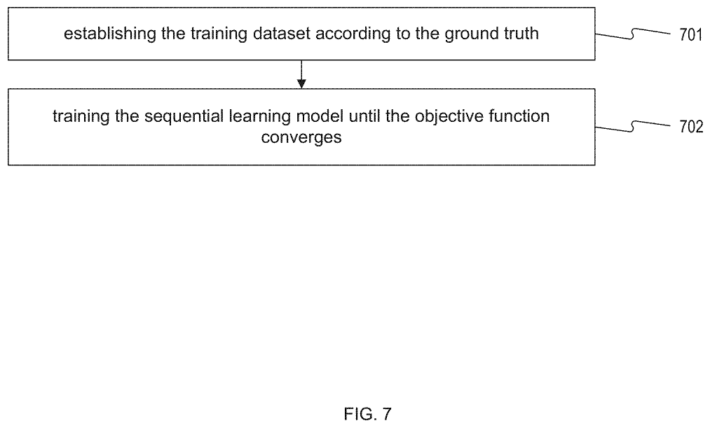

FIG. 7 illustrates the training process of the sequential learning model for both prediction of physiological-related parameters and estimation of the prediction uncertainty, according to an embodiment of the present disclosure. As shown in FIG. 7, at step 701, the training dataset may be established according to the ground truth. Particularly, a database of annotated training data with the ground truth of the sequence of physiological-related parameters and the uncertainty level may be assembled. In some embodiments, the ground truth might be available for all positions, or partial segments or only some locations in sequences. In some embodiments, the ground truth could be a single value for one position, or it could be multiple values (vector, matrix, and tensor) for one position. At step 702, based on the training dataset, the sequential learning model may be trained until the cost function (loss function) converges. The aim of training is to learn a mapping between the inputs and the outputs by finding the best fit between predictions made by the model and the ground truth values of those predicted parameters over the entire training space. Specifically, an end-to-end training may be performed by using optimization methods such as stochastic gradient descent method (SGD), RMSProp or Adam. Wherein, the loss function defined in any embodiment of present disclosure may be adopted here.

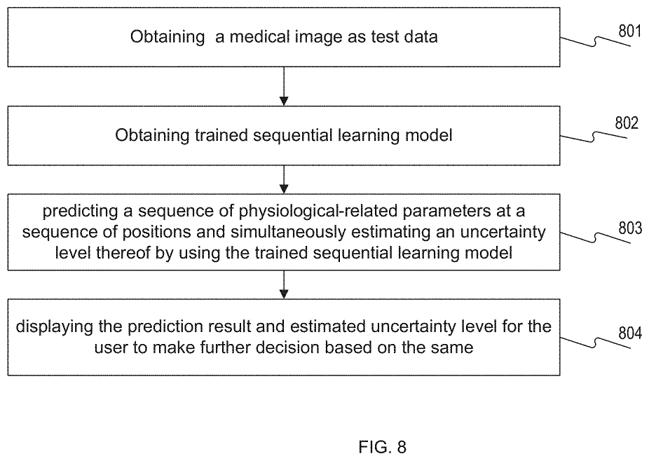

FIG. 8 illustrates the testing process of the trained sequential learning model for both prediction of physiological-related parameters and estimation of the prediction uncertainty, according to an embodiment of present disclosure. As shown in FIG. 8, at step 801, a medical image may be obtained as test data. As an example, the medical image may be obtained from an image acquisition device, a medical image database, or from a local storage, etc. At step 802, the trained sequential learning model may be obtained. Then, at step 803, a sequence of physiological-related parameters at a sequence of positions may be predicted and an uncertainty level thereof may be estimated simultaneously by using the trained sequential learning model based on the obtained medical image. At step 804, the prediction result and the estimated uncertainty level may be displayed to a user. In some embodiments, prompts may be provided to the user to make further decision based on the same. Examples of the further decision the user can take in consideration of the uncertainty level have been disclosed hereinabove and are not repeated here. As an example, the prompts may be visual, audible, and multi-media prompts, etc.

FIG. 9 illustrates a schematic block diagram of a system for predicting physiological-related parameters and the uncertainty level thereof based on a medical image, according to an embodiment of the present disclosure.

As shown in FIG. 9, the system may include a model training device 900a, an image acquisition device 900b and a parameter prediction and uncertainty estimation device 900c.

In some embodiments, the parameter prediction and uncertainty estimation device 900c may be a dedicated computer or a general-purpose computer. For example, the parameter prediction and uncertainty estimation device 900c may be a computer customized for the hospital to perform image acquisition and image processing tasks, or it may also be a server in the cloud.

The parameter prediction and uncertainty estimation device 900c may include at least one processor 903 configured to perform the method for predicting physiological-related parameter based on a medical image according to any embodiment of the present disclosure. The processor 903 may be configured to receive a medical image acquired by an image acquisition device 900b. The processor 903 may be further configured to predict a sequence of physiological-related parameters at a sequence of positions and simultaneously estimate an uncertainty level thereof from the medical image, by using a sequential learning model. The details of the method performed by the processor 903 are disclosed above and will not be repeated herein.

In some embodiments, the processor 903 may be processing device including one or more general processing device, such as microprocessor, central processing unit (CPU), graphics processing unit (GPU) and so on. More specifically, the processor may be complex instruction set computing (CISC) microprocessor, reduced instruction set computing (RISC) microprocessor, very long instruction word (VLIW) microprocessor, processor running other instruction sets or a combination of instruction sets. The processor 903 may also be one or more dedicated processing device, such as application specific integrated circuit (ASIC), field programmable gate array (FPGA), digital signal processor (DSP), system on chip (SoC) and so on.

The parameter prediction and uncertainty estimation device 900c may further include a storage 901, which may be configured to load or store the trained sequential learning model or an image prediction and estimation program according to any one or more embodiments of the present disclosure. The image prediction and estimation program, when implemented by the processor 903, may perform the method for predicting physiological-related parameters based on a medical image according to any embodiment of the present disclosure.

The storage 901 may be a non-transitory computer readable medium such as read only memory (ROM), random access memory (RAM), phase change random access memory (PRAM), static random access memory (SRAM), dynamic random access memory (DRAM), electrically erasable programmable read only memory (EEPROM), other types of random access memory (RAM), flash memory or other forms of flash memory, cache, register, static memory, compact disk read-only memory (CD-ROM), digital versatile disk (DVD) or other optical memory, cassette tape or other magnetic storage devices, or any other possible non-transitory medium for storing information or instructions accessible by computer devices and the like. When are implemented by the processor 903, the instruction stored on the storage 901 can perform the method for predicting physiological-related parameters based on a medical image according to any embodiment of the present disclosure.

Although the model training device 900a and the parameter prediction and uncertainty estimation device 900c are shown as independent devices in FIG. 9, in some embodiments, the parameter prediction and uncertainty estimation device 900c may also perform a model training function, and therefore, the storage 901 may be configured to load a set of training data labeled with the ground truth of the sequence of physiological-related parameters and the certainty level thereof, and the processor 903 may be configured to train the sequential learning model based on the loaded set of training data.

In some embodiments, the parameter prediction and uncertainty estimation device 900c may further include a memory 902 configured to load the sequential learning model according to any one or more embodiments of the present disclosure from such as the storage 901, or temporarily store intermediate data generated in the prediction and estimation procedure by using the sequential learning model. The processor 903 may be communicatively coupled to the memory 902 and configured to execute executable instructions stored thereon to execute the method for predicting physiological-related parameters based on a medical image according to any one of the embodiments of the present disclosure.

In some embodiments, the memory 902 may store intermediate information generated in the training stage or the inference stage, such as feature information, physiological-related parameters at individual positions, variances thereof, prediction results obtained for each time of Z sampling, the learning model parameters for each time of Z sampling, and each loss term value and the like generated while executing computer program, etc. In some embodiments, the memory 902 may store computer-executable instruction, such as one or more image processing programs. In some embodiments, the sequential learning model, each portion, layer, neurons (such as the latent random variables Z), and elements in the sequential learning model can be implemented as applications stored in the storage 901. These applications may be loaded into the memory 902, and then executed by the processor 903 to realize the corresponding processing.

In some embodiments, the memory 902 may be a non-transitory computer readable medium for storing information or instruction that can be accessed and executed by computer equipment and the like, such as read only memory (ROM), random access memory (RAM), phase change random access memory (PRAM), static random access memory (SRAM), dynamic random access memory (DRAM), electrically erasable programmable read only memory (EEPROM), other types of random access memory (RAM), flash disks or other forms of flash memory, cache, register, static memory or any other possible medium.

In some embodiments, the parameter prediction and uncertainty estimation device 900c may further include a communication interface 904 used for acquiring the medical image acquired by the image acquisition device 900b. In some embodiments, the communication interface 904 may include any one of network adapter, cable connector, serial connector, USB connector, parallel connector, high-speed data transmission adapter (such as optical fiber, USB 3.0, Thunderbolt interface, etc.), wireless network adapter (such as WiFi adapter), a telecommunication (such as 3G, 4G/LTE, 5G, etc.) adapter and so on.

The parameter prediction and uncertainty estimation device 900c may be connected to the model training device 900a, the image acquisition device 900b and other components via the communication interface 904. In some embodiments, the communication interface 904 may be configured to receive the trained sequential learning model from the model training device 900, and may also be configured to receive the medical image from the image acquisition device 900b.

In some embodiments, the image acquisition device 900b may include any one of general CT, general MRI, functional magnetic resonance imaging (such as fMRI, DCE-MRI and diffusion MRI), cone-beam computed tomography (CBCT), positron emission tomography (PET), single photon emission computed tomography (SPECT), X-ray imaging, optical tomography (OCT), fluorescence imaging, ultrasound imaging and radiation field imaging and the like.

In some embodiments, the model training device 900a may be configured to train a sequential learning model, and send the trained sequential learning model to the parameter prediction and uncertainty estimation device 900c, to predict physiological-related parameters and simultaneously estimate the uncertainty level based on a medical image using the trained sequential learning model. In some embodiments, the model training device 900a and the parameter prediction and uncertainty estimation device 900c may be implemented by a single computer or processor.

In some embodiments, the model training device 900a may be implemented using hardware specially programmed by software that performs training processing. For example, the model training device 900a may include a processor and a non-transitory computer readable medium similar to the parameter prediction and uncertainty estimation device 900c. The processor implements the training by executing the executable instructions of the training process stored in the computer readable medium. The model training device 900a may also include input and output interfaces to communicate with the training database, network, and/or user interface. The user interface is used to select the set of training data, adjust one or more parameters in the training process, select or modify a framework of the learning model.

Another aspect of the present disclosure is to provide a non-transitory computer readable medium storing instruction thereon, and when implemented, it causes one or more processors to perform the disclosed methods. The computer-readable medium may include volatile or nonvolatile, magnetic, semiconductor-based, tape-based, optical, removable, non-removable or other types of computer-readable media or computer-readable storage devices. For example, the computer-readable medium may be a storage device or a storage module in which a computer instruction is stored, as disclosed. In some embodiments, the computer-readable medium may be a magnetic disk or a flash drive on which computer instructions are stored.

Those skilled in the art may make various modifications and changes to the disclosed method, device, and system. In view of the description and practice of the disclosed system and related methods, other embodiments will be apparent to those skilled in the art.

It is intended that the description and examples are to be regarded as exemplary only, with the true scope being indicated by the appended claims and their equivalents.

What is claimed is:

1. A method for predicting physiological-related parameters based on a medical image, comprising:
receiving a medical image acquired by an image acquisition device; and
predicting, by a processor, a sequence of physiological-related parameters at a sequence of positions and simultaneously estimating an uncertainty level of the predicted sequence of physiological parameters from the medical image by using a sequential learning model, wherein the sequential learning model is trained to minimize a loss function associated with the uncertainty level.

2. The method of claim 1, further comprising:
allocating human resources for evaluation of the predicted sequence of physiological-related parameters based on the estimated uncertainty level thereof, wherein more human resources are allocated for evaluation when the uncertainty level is lower.

3. The method of claim 1, further comprising:
displaying the predicted sequence of physiological-related parameters and the estimated uncertainty level thereof in an associated manner for a user to make a further decision.

4. The method of claim 1, wherein the sequential learning model is a single sequential learning model, and the method further comprises:
receiving image patches or feature vectors extracted at the sequence of positions; and
predicting class labels, continuous physiological parameters, or segmentation masks at the sequence of positions as the sequence of physiological-related parameters and simultaneously estimating the uncertainty level of the predicted sequence of physiological-related parameters using the single sequential learning model.

5. The method of claim 4, wherein the physiological-related parameters are continuous physiological parameters, and the method further comprises:
predicting a sequence of mean physiological parameters together with a sequence of variances at the sequence of positions using the single sequential learning model;
outputting the predicted sequences of mean physiological parameters at the sequence of positions as the predicted sequence of physiological-related parameters; and
estimating the uncertainty level of the predicted sequence of physiological-related parameters based on the sequences of variances at the sequence of positions.

6. The method of claim 4, wherein the physiological-related parameters are class labels, and the method further comprises:
predicting a sequence of class labels together with a sequence of conditional probabilities at the sequence of positions using the single sequential learning model; and
estimating the uncertainty level of the predicted sequence of class labels based on the sequence of conditional probabilities at the sequence of positions.

7. The method of claim 5, further comprising:
randomly sampling latent variables in the sequential learning model multiple times;
for each sampled latent variable, predicting a corresponding sequence of physiological-related parameters;
determining the sequence of mean physiological parameters at the sequence of positions based on the sequences of physiological-related parameters predicted for the latent variables;
determining the sequence of variances at the sequence of positions based on the sequences of the physiological-related parameters predicted for the latent variables; and
estimating the uncertainty level based on the determined sequence of variances.

8. The method of claim 7, wherein the sequential learning model is a RNN model, and randomly sampling the latent variables in the sequential learning model further comprising: dropping out elements in neurons in one or more layers of the RNN model.

9. The method of claim 5, wherein the loss function comprises a squared L₂ norm loss based on the sequence of variances at the sequence of positions and a divergence between the sequence of mean physiological parameters and the sequences of ground truth physiological parameters.

10. The method of claim 9, wherein the sequential learning model is trained to minimize the divergence between the sequence of mean physiological parameters and the sequences of ground truth physiological parameters, or maximizing the sequence of variances at the sequence of positions.

11. The method of claim 6, wherein the loss function comprises the predicted sequence of class labels at the sequence of positions, wherein the sequential learning model is trained to minimize a divergence between a conditional probability and the corresponding class label.

12. The method of claim 1, wherein the medical image is a cardiovascular image, wherein the sequence of positions include the vessel centerline points,
wherein the physiological-related parameter includes at least one of vessel physiological-functional status, blood pressure, pressure drop, blood velocity, blood flow-rate, wall shear stress, fractional flow reserve (FFR), FFR change between adjacent vessel centerline points, instantaneous wave-free ratio (iFR), or iFR change between adjacent vessel centerline points.

13. A system for predicting physiological-related parameters based on a medical image, comprising:
a communication interface configured to receive a medical image acquired by an image acquisition device; and
a processor configured to predicting a sequence of physiological-related parameters at a sequence of positions and simultaneously estimating an uncertainty level of the predicted sequence of physiological parameters from the medical image by using a sequential learning model, wherein the sequential learning model is trained to minimize a loss function associated with the uncertainty level.

14. The system of claim 13, wherein the processor is further configured to:
allocate human resources for evaluation of the predicted sequence of physiological-related parameters based on the estimated uncertainty level thereof, wherein more human resources are allocated for evaluation when the uncertainty level is lower.

15. The system of claim 13, wherein the processor is further configured to:
display the predicted sequence of physiological-related parameters and the estimated uncertainty level thereof in an associated manner for a user to make a further decision.

16. The system of claim 13, wherein the sequential learning model is a single sequential learning model, and the processor is further configured to:
receive image patches or feature vectors extracted at the sequence of positions; and
predict class labels, continuous physiological parameters, or segmentation masks at the sequence of positions as the sequence of physiological-related parameters and simultaneously estimating the uncertainty level of the predicted sequence of physiological-related parameters using the single sequential learning model.

17. The system of claim 16, wherein the physiological-related parameters are continuous physiological parameters, and the processor is further configured to:
predict a sequences of mean physiological parameters together with a sequence of variances at the sequence of positions using the single sequential learning model;
output the predicted sequences of mean physiological parameters at the sequence of positions, as the predicted sequence of physiological-related parameters; and
estimate the uncertainty level of the predicted sequence of physiological-related parameters based on the sequences of variances at the sequence of positions.

18. The system of claim 16, wherein the processor is further configured to:
randomly sample latent variables in the sequential learning model multiple times;
for each sampled latent variable, predict a corresponding sequence of physiological-related parameters;
determine the sequence of mean physiological parameters at the sequence of positions based on the sequences of physiological-related parameters predicted for the latent variables;
determine the sequence of variances at the sequence of positions based on the sequences of the physiological-related parameters predicted for the multiple times; and
estimate the uncertainty level based on the determined sequence of variances.

19. The system of claim 17, wherein the loss function comprises a squared L₂ norm loss based on the sequence of variances at the sequence of positions and a divergence between the sequence of mean physiological parameters and the sequences of ground truth physiological parameters.

20. A non-transitory computer storage medium having computer executable instructions stored thereon, wherein the computer-executable instructions, when executed by a processor, perform a method for predicting physiological-related parameters based on a medical image, wherein the method comprises:
receiving a medical image acquired by an image acquisition device; and
predicting a sequence of physiological-related parameters at a sequence of positions and simultaneously estimating an uncertainty level of the predicted sequence of physiological parameters from the medical image by using a sequential learning model, wherein the sequential learning model is trained to minimize a loss function associated with the uncertainty level.
